(19) United States
(12) Patent Application Publication
(10) Pub. No.: US 2017/0231886 A1
(43) Pub. Date: Aug. 17, 2017

(54) METHOD AND COMPOSITION FOR IMPROVING SKIN HEALTH AND REDUCING VISIBLE SIGNS OF SKIN AGING

(71) Applicant: NUVOCARE HEALTH SERVICES INC., Toronto (CA)

(72) Inventor: Ryan Jasen FOLEY, Toronto (CA)

(21) Appl. No.: 15/431,031

(22) Filed: Feb. 13, 2017

Related U.S. Application Data
(60) Provisional application No. 62/295,604, filed on Feb. 16, 2016.

(51) Int. Cl. A61K 8/42; A61Q 19/08; A61K 8/66; A61K 8/97; A61K 8/65

(52) U.S. Cl. CPC A61K 8/42; A61K 8/97; A61K 8/65; A61K 8/66; A61Q 19/08

(57) ABSTRACT

The present description relates to a composition for treating skin aging in a subject comprising plant ceramides; collagen peptides; and a superoxide dismutase containing extract.

### CROSS-REFERENCE TO RELATED APPLICATIONS

The present application claims benefit of U.S. Provisional Application No. 62/295,604 filed Feb. 16, 2016, the content of which is hereby incorporated by reference in its entirety.

### TECHNICAL FIELD

The present relates to a composition for improving skin health and reducing skin aging.

### BACKGROUND ART

Skin changes are among the most visible sign of aging. The development of a wrinkle is one of the consequences of skin aging. A wrinkle, is a fold, ridge or crease in the skin. Age wrinkling in the skin is promoted notably by habitual facial expressions, aging, U.V. sun damage, smoking, poor hydration, and various other factors including nutritional deficiencies.

Skin is typically divided into three main parts. The outer part (epidermis) contains skin cells, pigments, and proteins. The middle part (dermis) contains blood vessels, nerves, hair follicles, and oil glands. The dermis provides nutrients to the epidermis. Finally, the inner layer (the subcutaneous layer) contains sweat glands, some hair follicles, blood vessels, and fat.

During normal aging processes, the epidermis thins and moisture levels decrease, even though the number of cell layers remains unchanged. Large pigmented spots may appear in sun-exposed areas. During aging, modifications in the connective tissue reduce the skin's strength and elasticity due to loss of collagen and elastin content. The blood vessels of the dermis become more fragile. The subcutaneous fat layer thins over time. Therefore, aging skin repairs itself more slowly. Accordingly, aging skin looks thinner, paler, clearer, and wrinkle appearance is more pronounced. Many of the causes of skin aging cited herein negatively impact levels of the two major components that make up healthy skin—collagen and elastin.

Known treatments for aging wrinkles include tretinoin, glycosaminoglycans, dermal fillers, botulinum toxin and laser resurfacing.

There is still a need to be provided with a composition or method for improving skin health and more particularly, for reducing signs of skin aging working through internal mechanisms using nutritional compounds.

### SUMMARY

In accordance with the present disclosure there is now provided a composition comprising plant ceramides; collagen peptides; and a superoxide dismutase containing extract.

In an embodiment, the plant ceramides are derived from wheat, rice, soy, potato, and/or spinach.

In another embodiment, the plant ceramides comprise phytoceramides and digalactosyl diglycerides.

In a further embodiment, the plant ceramides are Ceramosides®.

In another embodiment, the collagen peptides have an average molecular weight of about 2.0 kDa.

In another embodiment, the composition comprises up to 2.5-5.0 g of collagen peptides.

In a supplemental embodiment, the collagen peptides are Bioactive Collagen Peptides® (Verisol®).

In a further embodiment, the superoxide dismutase extract is from at least one of Cantaloupe melon (Cucumis meld subsp. var. cantalupo), Silphium perfoliatum L., Aronia melanocarpa, broccoli, cabbage, brussels sprouts, wheat sprouts, corn sprouts, soy sprouts, pea, and/or oat.

In a supplemental embodiment, the superoxide dismutase extract includes trace levels of Co-enzyme Q10, Alpha lipoic acid and catalase.

In another embodiment, the superoxide dismutase extract is EXTRAMEL®.

In a further embodiment, the composition described herein further comprises Vitamin C, Biotin, zinc, copper or a mixture thereof.

In a further embodiment, the composition described herein further comprises a Vitamin C source from at least one or more of ascorbyl palmitate, magnesium ascorbate and calcium ascorbate.

In an embodiment, the zinc is zinc glycinate.

In another embodiment, the copper is copper glycinate.

In a further embodiment, the composition described herein further comprises Dicalcium Phosphate, Croscarmellose Sodium, Microcrystalline Cellulose, Stearic Acid, Magnesium Stearate, Silicon Dioxide, Coating or a mixture thereof.

In an embodiment, the coating is at least one of Hypomellose, Glycerin, Beet Juice, Zinc oxide, Riboflavin and Stevia.

In another embodiment, the superoxide dismutase extract administered contains 140 IU superoxide dismutase per daily dosage.

In an additional embodiment, the composition comprises 30 mg/day to 70 mg/day. More particularly, the plant ceramides administered contain 15 mg of total phytoceramides and 12 mg of total digalactosyl diglycerides (DGDG) per daily dosage.

In another embodiment, the composition comprises 15 mg of plant ceramides comprising 7.5 mg of phytoceramides and 6 mg of digalactosyl diglycerides (DGDG).

In a further embodiment, the composition described herein consists of:

| | | |
|---|---|---|
| Protein | 1 | g; |
| Vitamin C | 180 | mg; |
| Biotin | 300 | μg; |
| Zinc | 15 | mg; |
| Copper | 1000 | μg; |
| Verisol ® Bioactive Collagen Peptides | 1250 | mg; |
| Ceramosides ® Plant Ceramide Extract | 15 | mg; and |
| EXRAMEL ® Cantaloupe Melon Extract | 5 | mg. |
| (providing Superoxide Dismutase) | | |

In an embodiment, the composition described herein is formulated as a caplet, a tablet, a capsule, or a powder.

In another embodiment, the composition described herein is for treating skin aging in a subject.

In accordance with the present disclosure there is also provided a method of treating skin aging in a subject comprising administering the composition described herein to the subject.

In an embodiment, the composition is administered twice daily.

In another embodiment, the composition is administered before a meal.

In another embodiment, the composition is administered 15 minutes before the meal.

In an additional embodiment, the following quantities are administered daily:

| | |
|---|---|
| Protein | 1.5 g-2.5 g; |
| Vitamin C | 270 mg-450 mg; |
| Biotin | 450 μg-750 μg; |
| Zinc | 22.5 mg-37.5 mg; |
| Copper | 1500 μg-2500 μg; |
| Bioactive Collagen Peptides | 1875 mg-5000 mg; |
| Plant Ceramide Extract | 22.5 mg-70.0 mg; and |
| Cantaloupe Melon Extract (providing | 7.5 mg-50 mg. |
| Superoxide Dismutase) | |

In an additional embodiment, the following quantities are administered daily:

| | | |
|---|---|---|
| Protein | 2 | g; |
| Vitamin C | 360 | mg; |
| Biotin | 600 | μg; |
| Zinc | 30 | mg; |
| Copper | 2000 | μg; |
| Bioactive Collagen Peptides | 2500 | mg; |
| Plant Ceramide Extract | 30 | mg; and |
| Cantaloupe Melon Extract (providing Superoxide | 10 | mg. |
| Dismutase) | | |

### DETAILED DESCRIPTION

It is provided a composition comprising specific nutrients that play a direct role in proactively supporting the health of the epidermal and dermal regions of the skin. The disclosed composition has the ability to neutralize specific free radicals that damage skin cell fibroblasts that are responsible for stimulating both collagen and elastin production.

Accordingly, it is provided a composition for improving skin health and reducing skin aging by working at all levels of skin physiology to improve skin moisture levels, stimulate collagen and elastin production and neutralize the accumulation of skin aging free radicals responsible for destroying the healthy youthful appearance of human skin cell complexion.

It is thus disclosed a composition that provides novel nutrients that specifically improve skin health at different levels of the skin while simultaneously controlling the specific free radicals that damage skin cell DNA.

In an embodiment, the composition described herein comprises plant ceramides, collagen peptides and a superoxide dismutase containing extract.

Ceramides are a type of sphingolipids which are essentially lipids substances present in cells. Ceramides are constituents of cells cytoplasmic membranes, naturally present in the skin. It has been shown that administration of ceramides at 30 mg/day to 70 mg/day (formulated as an oil), improved skin moisturization, skin barrier function, skin elasticity and skin micro relief in female subjects (Farcoderm, 2010, “Placebo-controlled, randomized and double blind clinical-instrumental evaluation of the efficacy of a diatery supplement in improving skin moisturization and skin micro-relief”, pp. 1-22). Plant ceramides derived from numerous sources, such as wheat, rice, soy and spinach, are encompassed herein to help support skin hydration and decrease transdermal water loss in the skin. In an embodiment, the plant ceramides encompassed herein are Ceramosides®. The plant ceramide extract encompassed herein can comprise phytoceramides and digalactosyl diglycerides. In an embodiment, the plant ceramide extract provided herein supplies 15 mg of total phytoceramides and 12 mg of total Digalactosyl diglycerides (DGDG) per daily dosage. Alternatively, the composition comprises 7.5 mg of phytoceramides and 6 mg of digalactosyl diglycerides (DGDG) taken twice daily.

Collagen peptides with a precise molecular weight help to simulate the cells in the skin to increase the production of both collagen and elastin. Administration of 2.5 g to 5.0 g of Collagen peptides to women aged 35-55 years showed improvement in skin elasticity levels and had a positive influence on skin moisture and skin evaporation (Proksch et al., 2014, Skin Pharmacol Physiol, 27: 47-55). Furthermore, an oral intake of 2.5 g of Bioactive Collagen Peptides (Verisol®) by women aged of 45-65 years showed reduced skin wrinkles and had positive effects on dermal matrix synthesis, particularly increased the content levels of procollagen type I and elastin (Proksch et al., 2014, Skin Pharmacol Physiol, 27: 113-119). In an embodiment, the collagen peptides encompassed herein have an average molecular weight of about 2.0 kDa. In a further embodiment, the collagen peptides present in the composition described herein are Bioactive Collagen Peptides (Verisol®).

Superoxide dismutase (SOD, EC 1.15.1.1) is an enzyme that alternately catalyzes the partitioning of superoxide (O₂₋) radicals into oxygen (O₂) or hydrogen peroxide (H₂O₂). Superoxide is produced as a by-product of oxygen metabolism and, if not regulated, causes many types of cell damage, Superoxide Dismutase derived from multiple sources such as for example cantaloupe melon (Cucumis melo L. subsp. var. cantalupo), Silphium perfoliatum L., Aronia melanocarpa extract, broccoli, cabbage, and brussels sprouts, wheat sprout extract, corn sprout extract, soy sprout extract, Pea extracts, and oat extracts are encompassed herein to help increase the skins natural defense mechanisms against the free radicals that damage skin cell DNA.

In an embodiment, the superoxide dismutase extract encompassed herein is EXTRAMEL®, consisting of a proprietary freeze-dried cantaloupe melon juice concentrate obtained by physical treatment (crushing of the melon, recovery of the pulp, centrifugation, filtration, freeze-drying) of a specific variety of melon (not a genetically modified organism) which contains high levels of SOD and other antioxidants (U.S. Pat. No 5,616,323). Administration of 10 mg of Extramel® (corresponding to 140 IU Superoxide Dismutase) daily to volunteers aged from 30-55 years, resulted in improved signs and symptoms of stress and fatigue linked to performance, physical (pain, sleep trouble), cognitive (concentration, weariness, sleep troubles) or behavioral (attitude, irritability, difficulty of contact) compared to placebo (Milesi et al., 2009, Nutrition Journal, 8: 1-7). Administration of SOD admixed with wheat gliadin showed protective effects against DNA damages, which coincided with reduced blood isoprostane levels (Muth et al., 2004, Free Radical Research, 38: 927-932).

Particularly, the melon superoxide dismutase extract encompassed herein comprises superoxide dismutase, Co-enzyme Q10, Alpha lipoic acid and catalase. In an embodiment, superoxide dismutase extract encompassed herein supplies 140 IU superoxide dismutase per daily dosage.

The composition described herein can further comprise Vitamin C, Biotin (Vitamin H), zinc in glycinate form and copper in glycinate form to support the body's ability to increase collagen and elastin production. The Vitamin C source can be from ascorbyl palmitate, magnesium ascorbate and/or calcium ascorbate.

In an embodiment, the composition further comprises Dicalcium Phosphate, Croscarmellose Sodium, Microcrystalline Cellulose, Stearic Acid, Magnesium Stearate, Silicon Dioxide, Coating (Hypomellose, Glycerin, Beet Juice, Zinc oxide, Riboflavin and Stevia).

In a particular embodiment, the composition described herein comprises:

| | | |
|---|---|---|
| Protein | 1 | g |
| Vitamin C | 180 | mg |
| Biotin | 300 | μg |
| Zinc | 15 | mg |
| Copper | 1000 | μg |
| Bioactive Collagen Peptides | 1250 | mg |
| Plant Ceramide Extract | 15 | mg |
| Cantaloupe Melon Extract (providing | 5 | mg |
| Superoxide Dismutase) | | |

More particularly, it is provided a composition named AGEOFF® glow comprising

| | | |
|---|---|---|
| Protein | 1 | g |
| Vitamin C | 180 | mg |
| Biotin | 300 | μg |
| Zinc | 15 | mg |
| Copper | 1000 | μg |
| Bioactive Collagen Peptides | 1250 | mg |
| Plant Ceramide Extract | 15 | mg |
| Cantaloupe Melon Extract (providing | 5 | mg |
| Superoxide Dismutase) | | |

More particulalry, the composition named AGEOFF® glow comprises ascorbyl palmitate, magnesium ascorbate and calcium ascorbate as Vitamin C source; Vitamin H as a Biotin source; zinc glucinate as a Zinc source, copper glycinate as a Copper source, Collagen Peptides with an average MW of 2.0 kD; 7.5 mg total of phytoceramides and 6 mg of digalactosyl diglycerides (DGDG); and 70 IU of superoxide dismutase.

It is provided a method of treating skin aging in a subject comprising administering the composition described herein to a subject. The composition described herein is in an embodiment formulated as a caplet, tablet, capsule or powder. In an embodiment, the composition described herein is administered twice daily, preferably 15 minutes before the first meal and 15 minutes prior to the last meal.

Accordingly, the composition and method described herein helps offset skin aging by working through internal mechanisms to improve skin health parameters in every level of the skin, by increasing skin moisture and decreasing water loss in the epidermis, assisting in the increase of collagen levels in the dermis region of the skin, and increasing elastin content in the dermis.

The combination disclosed herein is unique and different from any other combination because it is superior at working at every level of the skin to improve all skin health parameters. The composition encompassed herein decreases eye wrinkle volume appearance in addition to increasing skin collagen and elastin levels using intake of internal nutrients. In fact, the Bioactive Collagen Peptides (Verisol®) at the exact level provided in the preferred embodiment promoted a statistically significant reduction of eye wrinkle volume (p<0.05) in comparison to the placebo group after 4 and 8 weeks (20%) of intake. In addition, after 8 weeks of intake a statically significantly higher content of procollagen type I (65%) and elastin (18%) in the Bioactive Collagen Peptide(Verisol®) treated volunteers compared to the placebo-treated patients was detected.

In addition, the composition described herein is superior to other formulations because it also works through internal means in the epidermis of the skin to increase skin hydration and decrease transdermal water loss while also improving skin elasticity. The gluten-free plant ceramide extract used in the preferred embodiment (Ceramosides®) increases skin moisturization by 16% and 21% after 15 days and 60 days respectively. Transdermal water loss was also decreased by 13% and 14% after 15 days and 60 days respectively. Lastly, skin elasticity was significantly improved by 18% and 26% after 15 days and 60 days respectively.

Lastly, the composition disclosed herein works internally to protect skin cell DNA from damage. Cantaloupe Melon SOD extract taken internally and used in as disclosed herein as a preferred embodiment is able to protect against DNA damage, which coincided with reduced blood isoprostane levels.

The composition provided herein can be administered as an oral supplement that helps to protect collagen and elastin by protecting skin cell DNA.

While the invention has been described in connection with specific embodiments thereof, it will be understood that it is capable of further modifications and this application is intended to cover any variations, uses, or adaptations of the invention, including such departures from the present disclosure as come within known or customary practice within the art to which the invention pertains and as may be applied to the essential features hereinbefore set forth, and as follows in the scope of the appended claims.

What is claimed is:

1. A composition comprising:
plant ceramides;
collagen peptides; and
a superoxide dismutase containing extract.

2. The composition of claim 1, wherein the plant ceramides are derived from wheat, rice, soy, potato, and/or spinach.

3. The composition of claim 1, wherein the plant ceramides comprise phytoceramides and digalactosyl diglycerides.

4. The composition of claim 1, wherein the plant ceramides are Ceramosides®.

5. The composition of claim 1, wherein the collagen peptides have an average molecular weight of about 2.0 kDa.

6. The composition of any one of claim 1, wherein the collagen peptides are Bioactive Collagen Peptides (Verisol®).

7. The composition of claim 1, wherein the superoxide dismutase extract is from at least one of Cantaloupe melon ((Cucumis melo subsp. var. cantalupo)), Silphium perfoliatum L., Aronia melanocarpa, broccoli, cabbage, brussels sprouts, wheat sprouts, corn sprouts, soy sprouts, pea and oat.

8. The composition of claim 1, wherein the superoxide dismutase extract comprises trace levels of Co-enzyme Q10, Alpha lipoic acid and catalase.

9. The composition of claim 1, wherein the superoxide dismutase extract is EXTRAMEL®.

10. The composition of claim 1, further comprising Vitamin C, Biotin, zinc, copper or a mixture thereof.

11. The composition of claim 10, wherein the Vitamin C source is from at least one of ascorbyl palmitate, magnesium ascorbate and calcium ascorbate.

12. The composition of claim 10, wherein the zinc is zinc glycinate.

13. The composition of claim 10, wherein the copper is copper glycinate.

14. The composition of claim 1, further comprising Dicalcium Phosphate, Croscarmellose Sodium, Microcrystalline Cellulose, Stearic Acid, Magnesium Stearate, Silicon Dioxide, Coating or a mixture thereof.

15. The composition of claim 14, wherein the coating is at least one of Hypomellose, Glycerin, Beet Juice, Zinc oxide, Riboflavin and Stevia.

16. The composition of claim 1, wherein the composition consists of:

| | | |
|---|---|---|
| Protein | 1 | g; |
| Vitamin C | 180 | mg; |
| Biotin | 300 | μg; |
| Zinc | 15 | mg; |
| Copper | 1000 | μg; |
| Bioactive Collagen Peptides | 1250 | mg; |
| Plant Ceramide Extract | 15 | mg; and |
| Cantaloupe Melon Extract (providing | 5 | mg. |
| Superoxide Dismutase | | |

17. The composition of claim 1, said composition being formulated as a caplet, a capsule, a tablet or a powder.

18. The composition of claim 1, said composition comprising 15-70 mg of Plant Ceramide Extract; 1.25-5.0 g of Bioactive Collagen Peptides; and between 7.5-50 mg of Melon Extract (providing Superoxide Dismutase).

19. A method of preventing or treating skin aging in a subject comprising administering the composition of claim 1 to said subject.

20. The method of claim 19, wherein the following quantities are administered daily:

| | |
|---|---|
| Protein | 1.5 g-2.5 g; |
| Vitamin C | 270 mg-450 mg; |
| Biotin | 450 μg-750 μg; |
| Zinc | 22.5 mg-37.5mg; |
| Copper | 1500 μg-2500 μg; |
| Bioactive Collagen Peptides | 1875 mg-5000 mg; |
| Plant Ceramide Extract | 22.5 mg-70.0 mg; and |
| Cantaloupe Melon Extract (providing | 7.5 mg-50 mg. |
| Superoxide Dismutase) | |
